SEQUENCE LISTING (5 sequences)

SEQ ID NO: 1 - DNA, 434 bp, Artificial Sequence
  Control fragment sequence
caggaaacagctatgaccatgattacgccaagctatttaggtgacgcgttagaatactcaagctatgcatcaagcttggtaccgagctcggatccactagtaacggccgccagtgtgctggaattcaggcaagcagaagacggcatacgagatcgtgatgtgactggagttcagacgtgtgctcttccgatctctgcacaatgtgcacatgtaccctaaaacttagagtataataaaaataaaaaataaaaaaagaagtccaaaaaaagatcggaagagcgtcgtgtagggaaagagtgtagatctcggtggtcgccgtatcattcctgaattctgcagatatccatcacactggcggccgctcgagcatgcatctagagggcccaattcgccctatagtgagtcgtattacaattcactggccgtcgttttac

SEQ ID NO: 2 - DNA, 16 bp, Artificial Sequence
  M13F (-20) primer
gtaaaacgacggccag

SEQ ID NO: 3 - DNA, 17 bp, Artificial Sequence
  M13R primer
caggaaacagctatgac

SEQ ID NO: 4 - DNA, 57 bp, Artificial Sequence
  Control adapter (Top 5′)
ggaagcttgacattctggatcggtgactggagttcagacgtgtgctcttccgatctt

SEQ ID NO: 5 - DNA, 21 bp, Artificial Sequence
  Control adapter (Bottom 5′)
agatcggaagagcacacgtct

(19) United States
(12) Patent Application Publication
(10) Pub. No.: US 2022/0235412 A1
(43) Pub. Date: Jul. 28, 2022

(54) RAPID SEQUENCING OF SHORT DNA FRAGMENTS USING NANOPORE TECHNOLOGY

(71) Applicant: Samuel WILLIAMS, New York, NY (US)

(72) Inventor: Samuel WILLIAMS, New York, NY (US)

(21) Appl. No.: 17/391,737

(22) Filed: Aug. 2, 2021

Related U.S. Application Data
(63) Continuation of application No. 15/774,474, filed on May 8, 2018, now abandoned.
(60) Provisional application No. 62/254,579, filed on Nov. 12, 2015.

(51) Int. Cl. C12Q 1/6869; G16B 30/10; G16B 20/10; C12Q 1/6806; C12Q 1/686; G01N 33/487; G16H 10/40; G16H 70/00; G16B 20/20

(52) U.S. Cl. CPC C12Q 1/6869; G16B 30/10; G16B 20/10; C12Q 1/6806; G16B 20/20; G01N 33/48721; G16H 10/40; G16H 70/00; C12Q 1/686

(57) ABSTRACT

The disclosure described herein can be used for very rapid real-time acquisition of short DNA reads that can be used for time-sensitive aneuploidy detection in prenatal and IVF care as well as sequencing of small DNA fragments and amplicons in the field or clinic. This ability can expand the utility of nanopore-based sequencing methods for clinical and research applications.

### CROSS-REFERENCE TO RELATED APPLICATIONS

This application is a continuation of U.S. application Ser. No. 15/774,474, filed May 8, 2018, which is a national stage entry under 35 U.S.C. § 371 of International Application No. PCT/US2016/061859, filed Nov. 14, 2016, which claims the benefit of U.S. Provisional Application No. 62/254,579, filed Nov. 12, 2015, the disclosures of which are hereby incorporated by reference as if written herein in their entireties.

### FIELD OF THE DISCLOSURE

The field of this disclosure relates to library preparation and a data analysis method to enable rapid short-length DNA sequencing. In particular, it relates to a method to sequence short DNA fragments of DNA, in real-time, to enable the rapid diagnosis of aneuploidy or presence of genetic mutations in facilities outside of a laboratory.

### BACKGROUND OF THE DISCLOSURE

Nanopore-based sequencing records, in real-time, changes in electric current as an applied electric field drives single stranded DNA (ssDNA) through ˜500 nanopores assembled on the memory stick-sized device. The DNA library preparation and data analysis pipeline is designed to sequence and analyze, in parallel, ultra-long DNA fragments, as long as 100 kb in length. The purpose of assembling ultra-long DNA fragments have been for de novo genome assembly and non-reference scaffold building.

In the standard nanopore-based sequencing protocol, DNA is fragmented to an average length of >6 kb. DNA ends are then repaired, dA-tailed, and long DNA fragments are ligated to a kit adapter mix. The adapter mix consists of two DNA adapters: a Y-shaped adapter and a hairpin-shaped adapter. The Y-shape adapter has a leader strand that guides DNA to the nanopore, and a pre-attached ES protein that separates the complimentary DNA strands and aids the passage of DNA through the pore. The hairpin shaped adapter enables a “U-turn” at the hairpin and continued sequencing of the complementary strand of a double strand DNA (dsDNA). The structure of the Y adapter/template/hairpin-adapter allows the sequencer to generate a template read, a complementary read, and a calibration of these two reads, (i.e., a 2D read for dsDNA). 2D reads improve sequencing quality from a single dsDNA molecule. A His-Tagged E3 protein, attached to the hairpin-shaped adapter during the ligation process, slows sequencing speeds and is used for purification of DNA fragments ligated to the hairpin adapter using His-Tag bead purification. The parallel sequencing capacity of MinION, Oxford Nanopore Technologies,(˜500) is much lower than several other sequencing platforms. (MiSeq, Illumina 25×10⁶; Ion Proton, Life Technologies, 80×10⁶). However, the MinION platform sequences individual nucleotides at a much faster rate (1200-1800 nt/min), compared to Ion Proton and MiSeq, respectively (1 nt/min and 0.17 nt/min).

### SUMMARY OF THE DISCLOSURE

Nanopore-based sequencing has the distinct advantages that after completing sequencing of one DNA fragment, the DNA sequencing of another DNA fragment begins, and reads are generated in real-time so sequencing can be stopped when sufficient reads are obtained.

The current MinION nanopore genomic DNA library preparation and sequencing protocols cannot be used for short fragment library preparations. The disclosure described herein relates to a library preparation and a data analysis method to enable rapid short length DNA sequencing.

In one embodiment, the disclosure provides a nanopore-based sequencing method to generate many fold reads in a given time compared with long-fragment sequencing.

In another embodiment, the disclosure provides a nanopore-based sequencing method on a biological sample which comprises detecting the presence of a nucleic acid of fetal origin in the biological sample.

In yet another embodiment, the disclosure provides a nanopore-based sequencing method for prenatal diagnosis. The term “prenatal diagnosis” as used herein covers determination of any fetal condition or characteristic which is related to the fetal DNA sequenced by the nanopore-based sequencing method described herein.

In another embodiment of this disclosure comprises a nanopore-based sequencing method for sex determination and detection of fetal abnormalities, which may include, but are not limited to, chromosomal aneuploidies or simple mutations.

In yet another embodiment of the disclosure are nanopore-based sequencing methods for rapid detection and phenotyping of pathological agents.

The disclosure described herein enables a wide range of new research and clinical applications which can be performed in physician's offices and field settings.

### BRIEF DESCRIPTION OF THE DRAWINGS

FIG. 1A. Schematic of the short-fragment sequencing library preparation. dsDNA is fragmented, size selected, end repaired, and coOncentrated. Increased concentrations of Y-shape adapters with attached E5 proteins and hairpin adapters are ligated onto the dsDNA and E3 proteins (green) bind to hairpin adapters. Electric current then drives a single strand of DNA through the nanopore (light gray).

FIG. 1B. Optimization of short-fragment Library preparation. Lane 1, control DNA fragment; lane 2, ligation of control fragment and adapters using manufacturer's protocol; lanes 3-7, incremental improvements in ligation efficiency using purification of fragmented and dA-tailed template DNA (lane 3), reduced reaction volume (lane 4), incorporation of a 1-2 hour incubation at 4° C. (lanes 5, 6) and reducing RT incubation time to 5 min in order to reduce release of E5 proteins from adapters (lane 7).

FIG. 2A. Use of short-DNA fragment sequencing using Minion was able to correctly determine gender and detect aneuploidy in DNA samples from a normal male and female, a female with monosomy X, a male with trisomy 12, and a male with trisomy 21 (p<0.001). The copy number of each chromosome was reflected by the corrected normalized percentage of UA (Norm′_% UAᵢ). Black dots represent chromosomes without significant copy number changes; red dots represent chromosomes with significant copy number changes comparing to a normal male reference; dotted line represent 99.9% confidence intervals.

FIG. 2B. Theoretical lower unique alignment (UA) required for aneuploidy detection under Poisson distribution. When λ=41, p(x>1.5λ)=0.0008. pβ(x′<1.25 k)=0.10.

FIG. 2C. Theoretical lower detection power using the 15K reference under Poisson distribution. The Y chromosome has fewest UA, 79-80, assigned. When λ=79, p(x>1.5λ)=1.07×10⁻⁵. p_{β}(x′<1.25λ)=0.034.

FIG. 2D. Sequencing yield of a short-fragment library across time showing raw reads, 2D reads, and reads uniquely aligned to Hg19 reference genome.

FIG. 3. MinION library preparation.

FIG. 4. Software comparision.

FIG. 5. MinION Run Summary.

FIG. 6. Comparison of the 15K normal male reference and the GRCh37 human reference genome.

FIG. 7. ULCS cytogenetics analysis.

FIG. 8. Internal normalization. Runs 1-4, using an internal reference, has a very low coefficient of variation, whether using our own DNA sequencing data or that obtained from other groups.

### DETAILED DESCRIPTION OF THE DISCLOSURE

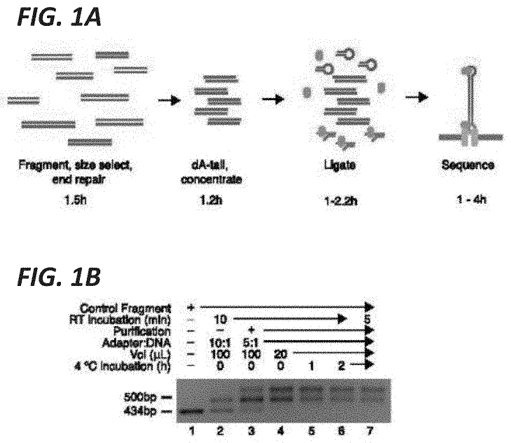

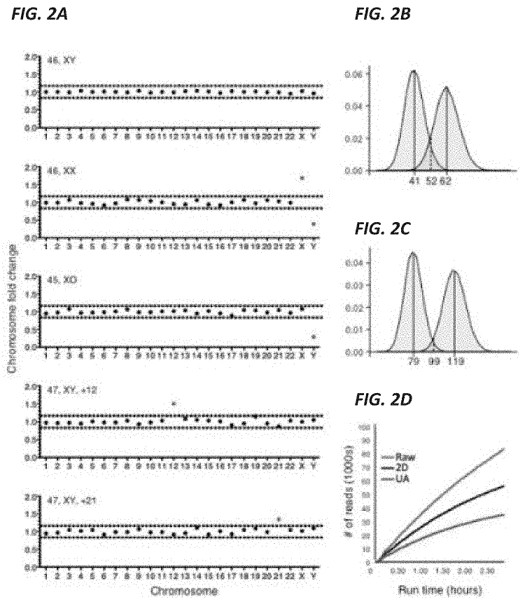

To maintain equivalent molar concentrations for short DNA fragment-length library preparations compared with long fragment-length, ˜18-fold lower total ng of input DNA and improved ligation efficiency was required (FIG. 1B). We systematically modified the protocol to improve ligation efficiency. To monitor ligation reactions, a 434 bp PCR product and a 57 bp control adapter duplex with a T-overhang were used (Table 1).

TABLE 1
Sequence Information

| | | |
|---|---|---|
| SEQ ID | Control | CAGGAAACAGCTATGACCATGATTAC |
| NO: 1 | fragment | GCCAAGCTATTTAGGTGACGCGTTAGA |
| | sequence, | ATACTCAAGCTATGCATCAAGCTTGGT |
| | 434 bp | ACCGAGCTCGGATCCACTAGTAACGGC |
| | | CGCCAGTGTGCTGGAATTCAGGCAAGC |
| | | AGAAGACGGCATACGAGATCGTGATG |
| | | TGACTGGAGTTCAGACGTGTGCTCTTC |
| | | CGATCTCTGCACAATGTGCACATGTAC |
| | | CCTAAAACTTAGAGTATAATAAAAATA |
| | | AAAAATAAAAAAAGAAGTCCAAAAAA |
| | | AGATCGGAAGAGCGTCGTGTAGGGAA |
| | | AGAGTGTAGATCTCGGTGGTCGCCGTA |
| | | TCATTCCTGAATTCTGCAGATATCCAT |
| | | CACACTGGCGGCCGCTCGAGCATGCAT |
| | | CTAGAGGGCCCAATTCGCCCTATAGTG |
| | | AGTCGTATTACAATTCACTGGCCGTCG |
| | | TTTTAC |
| SEQ ID | M13F (−20) | GTAAAACGACGGCCAG |
| NO: 2 | primer | |
| SEQ ID | M13R primer | CAGGAAACAGCTATGAC |
| NO: 3 | | |
| SEQ ID | Control | GGAAGCTTGACATTCTGGATCGGTGAC |
| NO: 4 | adaptor | TGGAGTTCAGACGTGTGCTCTTCCGAT |
| | (Top 5′) | CTT |
| SEQ ID | Control | |
| NO: 5 | adaptor | |
| | (Bottom 5′) | AGATCGGAAGAGCACACGTCT |

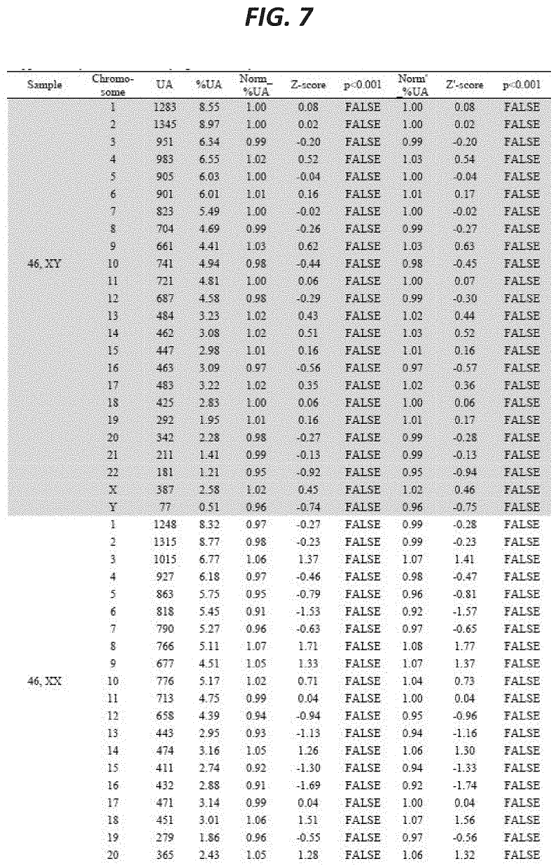

Use of the manufacturer's protocol resulted in <5% of all end products having two adapters attached (FIG. 1B, lane 2). By purifying dA-tailed DNA prior to ligations, the percentage of end products having two adapters ligated increased to 25% (FIG. 1B, lane 3). Reducing reaction volumes from 100 μto 20 μfurther increased the percentage of end products with two adapters ligated to 48% (FIG. 1B, lane 4). By combining a 10 min RT and 1-2 h at 4° C. incubation, we were able to increase the percentage of fragments with adapters ligated to both ends to 61-63% (FIG. 1B, lanes 5-7) without releasing the pre-attached ES protein. Thus, by purifying and then concentrating dA-tailed DNA to reduce the reaction volume and introducing a prolonged 2 h ligation at 4° C., we increased the percentage of final products with adapters ligated to both ends from <5% to 63% (FIG. 1B lane 2 vs 7) and provided sufficient materials for downstream His-Tag bead purification (FIG. 3).

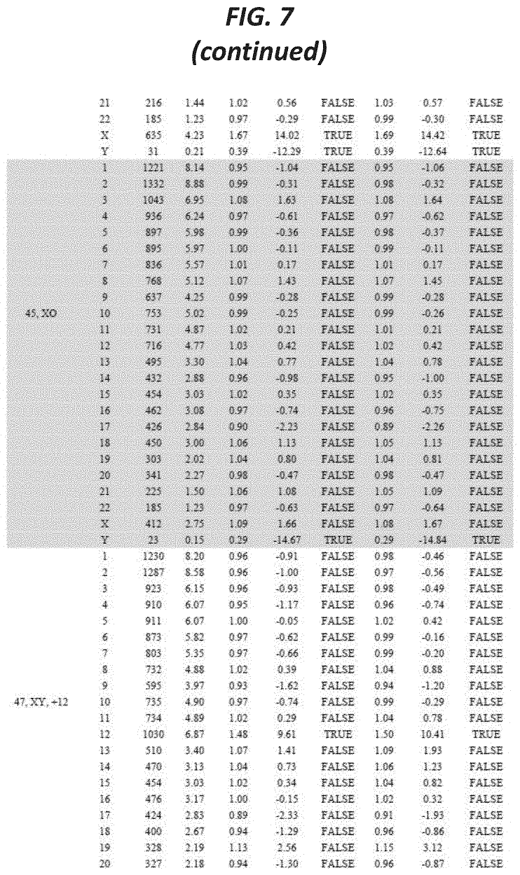

To determine the optimal tool for data analysis of the increased number of reads obtained with sequencing of short DNA, we compared LAST—an alignment program recommended by MAP—with two similar programs, Bowtie2 and Blat (8-10), using a training library generated through a MinION short DNA sequencing run (FIG. 4). While Bowtie2 and LAST completed alignments more quickly (1 min and 14 min, respectively) than Blat (68 min), Blat generated more good alignments (65%) compared with Bowtie2 and LAST (58% and 61%, respectively) for the same datasets, likely due to the tendency for MinION sequencing errors resulting in deletions (FIGS. 3-4). Blat also generated more unique alignment (62%) compared with Bowtie2 and LAST (45% and 55%, respectively). Blat was used for alignment of the MinION short DNA sequencing results to provide the most inclusive alignment results. Given sufficient computational resources on a high performance server, increasing parallel threats can further reduce the run time.

To demonstrate clinical utility of nanopore-based sequencing of short DNA fragments, we tested the ability of this approach to diagnose aneuploidy. Fetal aneuploidy testing is routinely performed as a component of prenatal testing (e.g. amniocentesis, chorionic villus sampling (CVS)), preimplantation genetic screening (PGS) of embryos in in-vitro fertilization (IVF) and evaluation of miscarriage tissue. A rapid diagnosis is clinically vital in order to enable timely management. In the case of prenatal samples obtained through an amniocentesis or CVS, rapid results will enable treatment before the pregnancy progresses to a more advanced gestational age when treatment options are more limited, technically difficult and dangerous to the mother. In the case of PGS, rapid testing will enable transfer of the embryo in a given IVF cycle without the need to freeze embryos. However, standard methods to diagnose aneuploidy, such a karyotyping and microarray analysis, take 7-21 days to complete. Ultra-low coverage sequencing (ULCS) for detection of aneuploidy is a new strategy for whole-genome aneuploidy detection that requires alignment of reads to a reference genome assembly to assess for aneuploidy but still requires 15-21 h to complete and requires costly and technically advanced library preparation and sequencing platforms that cannot be readily used in a physician's office or in low complexity settings. The ULCS approach for determining aneuploidy requires that the reads need only be sufficiently long to enable unique alignment to the genome. Thus, a method to rapidly sequence large numbers of short DNA fragments in real-time would enable rapid diagnosis of aneuploidy in settings outside of an advanced laboratory facility.

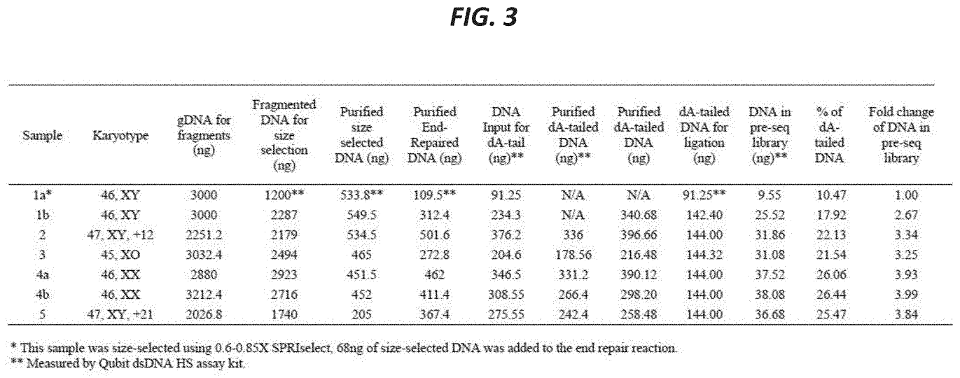

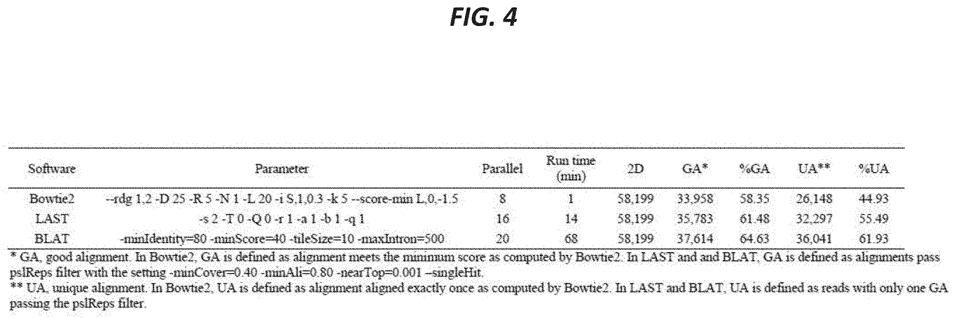

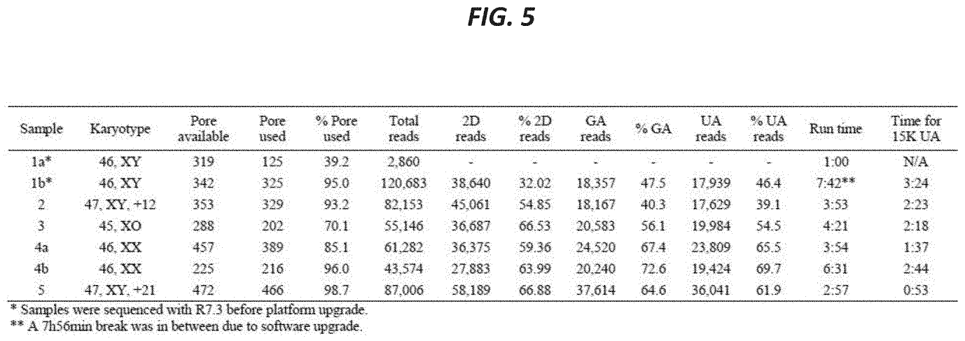

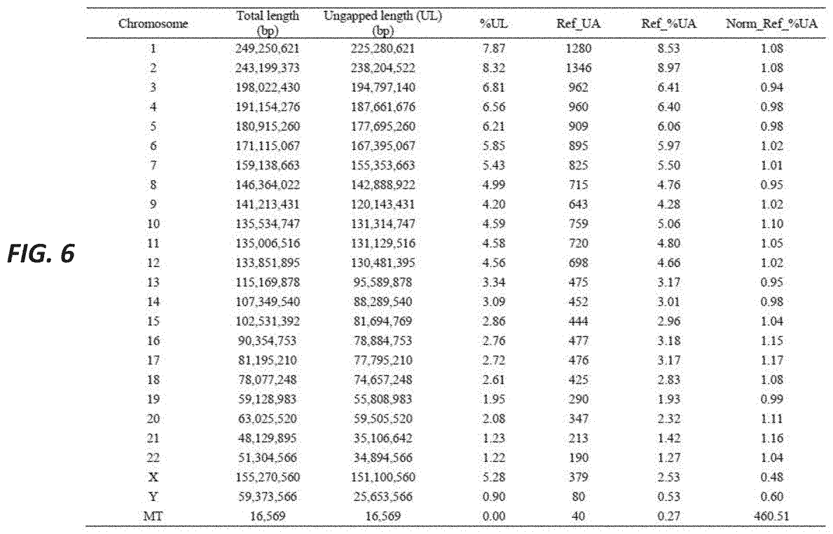

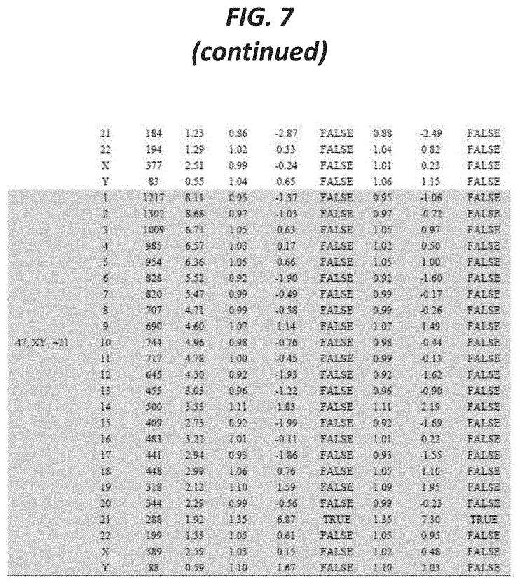

Purified genomic DNA samples from a normal male and female, a male with trisomy 12, a male with trisomy 21 and a female monosomy X were fragmented, size-selected (350-600 bp), and processed as described (FIG. 3). Sequencing short DNA fragment libraries prepared using our protocol with MinION generated ˜500 unique reads after the first 3 min of sequencing and 43-87K raw reads and 27-58K 2D reads (32-67%) after 4 hours of sequencing (FIG. 2, FIG. 5). This compares favorably to the traditional MinION sequencing protocol that sequenced fewer than 12,000 reads after 36 h. Of the reads generated using our protocol, 40-70% of the 2D reads could be uniquely mapped to one location (FIG. 5).

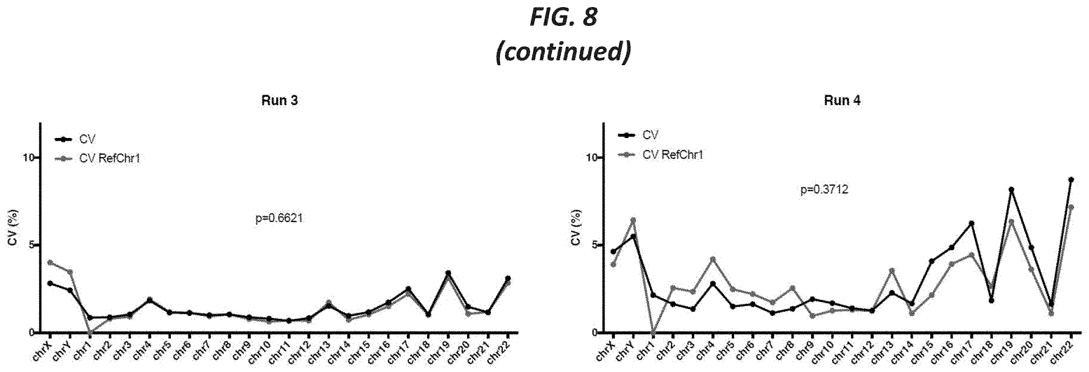

Using the short fragment length DNA sequencing library preparation and analysis pipeline we obtained sufficient numbers of reads for successful determination of gender and aneuploidy (p<0.001) in all samples within 2-4 h (FIG. 2A). By Normal approximation of Poisson distribution, the chance of a type II error for detecting aneuploidy (pβ-aneuploidy) was <0.05 (FIG. 2C, FIG. 7). As MinION is easily scalable, cytogenetic analysis can be done within 1-2 h by running two MinION sequencers in parallel and 30 min-1 h by running four MinION sequencers in parallel.

In summary, in addition to the intended role of MinION for sequencing long fragments of DNA, our results demonstrate that MinION can also be used for very rapid real-time acquisition of short DNA reads that can be used for time sensitive aneuploidy detection in prenatal and IVF care as well as sequencing of small DNA fragments and amplicons in the field or clinic. This ability can expand the utility of the MinION into new clinical and research applications.

The disclosure will now be illustrated in the following Examples, which do not in any way limit the scope of the invention.

### EXAMPLES

### Example 1

### Development of Ligation Conditions

To assess the ligation efficiency, a short DNA control fragment were used for initial ligation reactions. The fragment was generated using PCR with M13 forward and reverse primers to amplify a 434-bp fragment from a pCR-Blunt vector using Q5 High-Fidelity DNA Polymerase (NEB). See Table 1.

A 50-ml PCR reaction was prepared following the manufacturer's protocol. The PCR reaction was subjected to a 30-sec initial denaturation at 98° C., 25 cycles of 10-sec denaturation at 98° C., a 30-sec annealing at 57° C., and a 20-sec elongation at 72° C. A final elongation step at 72° C. for 2 min was added to ensure complete amplification. The PCR product was purified using a QlAquick PCR Purification Kit following the manufacturer's protocol. A 57-bp asymmetric adapter with a T overhang was used as a control adapter to assess ligation efficiency See Table 1. The control adapters were diluted to 0.4 mM in MinION adaptor buffer (50 mM NaC1 and 10 mM Tris-HC1, pH 7.5) to simulate the 0.2-mM concentration of the Y shaped and hairpin adapters in the adaptor mix (Oxford Nanopore).

Ligation reactions were initially performed following the MinION Genomic Sequencing Kit protocol (Oxford Nanopore, SQK-MAP004). Control DNA fragments (0.2 pmol, 52 ng) were added to a 30 μl NEB Next dA-Tailing Module (NEB) reaction [4 ml of control fragments, 21 μl of Qiagen Buffer EB, 3 μl of 103 NEB Next dA-tailing reaction buffer, and 2 μl of Klenow fragments (3′→5′ exo-)]. Reactions were performed at 37° for 30 min in a Bio-Rad C1000Touch Thermal Cycler. All the dA-tailing reactions were added to a total volume of 100 μl [30 μl of dA-tailing reaction, 10 μl of control adapter, 10 μl of nuclease-free water, 50 μl of NEB Blunt/TA Ligase Master Mix (NEB)] and incubated at room temperature (23-25° C.) for 10 min.

Because so few control fragments had adapters ligated on both ends (FIG. 1B, lane 2), an alternative Klenow fragment (39/59 exo-) (NEB) was used for dA tailing, and the dA-tailing reactions were purified before being added to the ligation reactions. Control DNA fragments (250 ng) were subjected to a dA-tailing reaction [2.5 μl of NEBuffer II, 5 ml of 1 mM deoxyadenosine triphosphate (dATP), 1 ml of Klenow fragment (39/59 exo-), and nuclease-free water to a total volume of 25 μl ]. After purification with 1.8-fold AMPure XP beads (Beckman Coulter following the manufacturer's protocol for the SPRI select reagent (Beckman Coulter), the dA-tailed control fragment was eluted in 12 μl of ⅕ Qiagen Buffer EB (2 mM Tris-C1, pH 8; Qiagen) and diluted to 0.05 mM (13 ng/ml).

Overnight ligation reactions at 16° C. using T4 DNA ligase (NEB) to ligate a 10:1 adapter-fragment mixture (4 pmol control adapter, 0.2 pmol control fragment in 2 μl 10×T4 DNA ligase buffer, 1 ml T4 DNA ligase, and NF H₂O to 20-μl final volume) resulted in ˜75% of the control fragments having adapters on both ends, which would not be sufficient final products for downstream steps. Therefore, the reactions were run in duplicate and combined. Then 5:1 ratios were used to preserve the adapters provided in the MinION kits.

The second ligation reactions were a replication of the manufacturer's ligation protocol using the purified dA-tailed DNA, as described previously (FIG. 1B, lane 3), using 100 μl of ligation reaction with 0.4 pmol of DNA, 26 μl of Buffer EB, 10 μl of control adapter, 50 μl of Blunt/TA Ligase MasterMix (NEB), and 10 μl of nuclease-free water (Ambion). Reactions were incubated at room temperature for 10 min and purified using 1.8-fold AMPure XP beads, washed with the wash buffer in the SQK-MAP003MinION Genomic DNA Sequencing Kit (750 mM NaCl, 10% PEG 8000, 50 mM Tris-HC1, pH 8.0), and eluted in 20 μl of Buffer EB.

The third ligation reactions were a reduced-volume system using purified dA-tailed DNA, as described previously (FIG. 1B, lanes 4-7). A 20-ml ligation reaction containing 0.2 pmol of DNA (4 ml), 2 pmol of control DNA adaptor (5 μ1), 10 μl of Blunt/TA Ligase Master Mix, and 1 μl of nuclease-free water was incubated for 10 min at room temperature, purified using one-fold AMPure XP beads with the SQK-MAP003 wash buffer, and eluted in 20 μl of Buffer EB (FIG. 1B, lane 4). Reactions were carried out at room temperature for 5-10 min, followed by 4° C. incubation for 1-2 hr (FIG. 1B, lanes 5-7). Reactions were purified using one-fold AMPure XP beads with SQK-MAP003 wash buffer and eluted in 20 μ1 of Buffer EB. Purified ligation products were run on 2% agarose gels. Portions of the ligation products were estimated using ImageJ densitometry analysis with two technical replicates.

### Example 2

### Nucleic Acid Manipulations

To facilitate maximum recovery of material, 1.5-ml low-retention microcentrifuge tubes and low-retention tips were used unless stated otherwise. For all reactions performed in a thermal cycler, 0.2-ml PCR tubes were used (Axygen). An Agencourt SPRIStand Magnetic 6-tube Stand (Beckman Coulter) was used for pelleting of SPRI select and AMPure XP bead-related purification; a DynaMag-2 magnet (Life Technologies) was used for His-tag bead isolation.

### Example 3

### Genomic DNA Samples

Genomic DNA (gDNA) samples from a karyotypically normal male and female, a male with trisomy 12, a male with trisomy 21, and a female with monosomy X were used for cytogenetic analysis using short-DNA-fragment ULCS with the MinION. Blood B-lymphocytes from karyotypically normal human male and female samples were obtained from the Coriell Institute Cell Repositories (GM12877 and GM12878) and cultured according to the protocol provided by the Coriell Institute. gDNA was extracted from cell cultures from the second passage using a QlAamp Blood DNA Mini Kit (Qiagen) following the manufacturer's manual. gDNA from a male with trisomy 21 was provided by the Coriell Institute Cell Repositories (NG05397). DNA samples from a male with trisomy 12 and a female with monosomy X were obtained from the products of conception of miscarriage cases that had cytogenetic testing performed using G-band karyotyping. gDNA was extracted using an All Prep DNA/RNA/Protein Mini Kit (Qiagen) from the trophoblastic primary cell cultures of the chorionic villus. The quality of gDNA was examined on 0.8% agarose gel and quantified using a NanoDrop 1000 Spectrophotometer (Thermo Fisher Scientific). DNA was stored at −20° C. until needed.

### Example 4

### Library Preparation

For library preparation, 120 μl of 25 ng/ml gDNA in TE Buffer (pH 8.0) was fragmented using a Covaris 5220 focused ultra-sonicator at the manufacturer's 500-bp setting in micro-TUBEs (Covaris). For size selection, 100 μl of fragmented gDNA was used. Size selection was performed in a 1.5-ml DNA LoBind tube (Eppendorf) using SPRIselect reagent following the manufacturer's double-sized selection protocol using a right-side 0.55 times, left side 0.7 times setting (Beckman Coulter). DNA was eluted in 40-50 μl of Buffer EB in a 1.5-ml DNA LoBind tube. Then 2 μl of DNA was used for a 2% gel electrophoresis to confirm fragment size. Purified DNA (3 μl ) was saved for NanoDrop quantification. Size-selected DNA fragments were ˜350-600 bp in length.

Buffer EB was added to size selected DNA to a final volume of 80 μl . End-repair reactions were performed using a NEB Next End Repair Module (NEB) in a 1.5-ml DNA LoBind tube. Then 5 μl of DNA CS (Oxford Nanopore, SQK-MAP004), 10 μl of 10 x NEB Next End Repair Reaction Buffer, and 5 μl of NEB Next End Repair Enzyme Mix were added to the size-selected DNA fragment and mixed by gently pipetting. The reactions were incubated at room temperature for 25 min and purified using 1.8-fold AMPure XP beads following the SPRI select reagent protocol in a DNA LoBind tube. The end-repaired DNA was eluted in 22 μl of Buffer EB, and the DNA was quantified using a Qubit dsDNA HS AssayKit (Life Technologies).

End-repaired DNA was subjected to a dA-tailing reactionusing a Klenow fragment (3′→5′ exo-) in a total volume of 25 μl in a sterile PCR tube. The reaction contained 2.5 μl of NEBuffer II, 1 μl of Klenow fragment (3′→5′ exo-), 16.5 μl of end-repaired purified DNA, and 5 μl of dATP (1 mM). Reactions were incubated in a Bio-Rad C1000 Thermal Cyclerat 37° C. for 45 min, purified using 1.8-fold AMPure XP beads, and then eluted in 12 μl of ⅕ Buffer EB. The purified product was quantified using NanoDrop and a Qubit dsDNA HSAssay Kit (Life Technologies) and diluted to ˜0.05 mM (˜18 ng/ml) with ⅕ Buffer EB to be used as the dA-tailed DNA in subsequent reactions.

His-tag Dynabeads (10 ml) (Invitrogen) were washed in 1.5-ml low-retention tubes in a MinION Genomic DNA Sequencing Kit following the manufacture's protocol on a DynaMag-2 magnetic stand (Invitrogen). Washed beads were resuspended in 40 μl of undiluted wash buffer (SQK-MAP004) and kept on ice. Ligation reactions were performed in a 1.5-ml low-retention tube. Twenty-microliter reactions contain 4 μl of dA-tailed DNA (0.2 pmol), 5 μl of adaptor mix (1 pmol) (SQK-MAP004), 1 μl of HP adapter (1pmol) (SQK-MAP004), and 10 μl of Blunt/TA Ligase Master Mix (NEB). The reactions were mixed by pipetting gently between each sequential addition and spun down briefly in a benchtop centrifuge. Ligation reactions were incubated at room temperature for 5 min follow by 4° C. for 2 hr. For each sample, 2×20 μl reactions were performed in separate tubes and combined for His-tag bead purification.

In 1.5-ml low-retention tubes, 40 μl of washed His-tag beads were added to the adapter-ligated DNA and carefully mix by gentle pipetting. The mixture was incubated at room temperature for 5 min and placed on ice for 30 sec. His-tag bead purification was performed following the protocol of the MinION Genomic DNA Sequencing Kit (SQK-MAP004). Pelleted beads were resuspended 28 μl of the ELB elution buffer (SQK-MAP004) by gently pipetting 10 times. The suspension was incubated at room temperature for 5 min and placed on ice for 30 sec, and this was repeated once before placing the suspension back on the magnetic rack for pelleting. The eluate was transferred to a clean 1.5-ml low-retention tube, incubated on ice for 30 sec, and then placed on a magnetic rack for 2 min for pelleting any residual beads. The eluate then was carefully transferred to a 1.5-ml low-retention tube. This library was called the presequencing mix. Then 4 μl of the presequencing mix was used for quantification by a Qubit dsDNA HS Assay Kit.

### Example 5

### MinION Sequencing

Then 150 ml of the priming mix (147 μl of EP buffer and 3 μl of fuel mix) was loaded on a MinION Flow Cell (R7.3) and incubated for 10 min. The priming process was repeated once. Then 150 μl of the MinION sequencing library (12 μl of the presequencing mix, 135 ml of EP buffer, and 3 ml of fuel mix) was gently mixed and loaded to the MinION Flow Cell. The MAP 48-hr gDNA sequencing protocol was used, and the sequencing reaction was stopped when sufficient data were collected.

### Example 6

### Data Analysis

Metrichor Agent V2.26 was used to transfer local fast5 files, and 2D Base calling Rev1.14 was used to convert currency into base events (Oxford Nanopore Technologies). Pore tools v0.5.0 was used to convert Fast5 to fastQ files. The first and last 50 bases were removed from each sequence using cut adapt v1.7.1, and sequences that were at least 50 bases long were kept after the removal. Both 1D and 2D reads were aligned to the Ensembl GRCh37 human reference genome using BLAT (FIG. 3).

Less than 1% of 1D sequences passed the screening criteria (covers >40% of query, >80% alignment identity) and consequently only 2D sequences were used for further analysis. 2D reads with a unique alignment match (UA) to a genomic location were retained for further analysis. Bowtie2 was also tested for mapping 2D sequences to a human reference genome. As Bowtie2 was designed for high-throughput mapping of short sequences (50-200bp), <5% full length 2D reads could be mapped. Bowtie2—bwa-sw-like settings developed for 454 data were also tested, only 36% of the 2D reads were UA. Therefore, we used Bowtie2 to align the first 200 bp of the 2D reads, and generated 45% UA in ˜1 min (FIG. 4). 2D reads were also mapped to the reference genome using LAST using the recommended setting that were reported to be most inclusive for alignment for MinION long reads, however, it produced fewer UA comparing to the BLAT pipeline using the same screening criteria (FIG. 3). Hence, only the UA from the BLAT pipeline were used for the fast cytogenetic analysis using the ultralow coverage sequencing (ULCS).

### Example 7

### Digital Karyotyping using Ultra Low Coverage Sequencing (ULCS)

Ultralow coverage sequencing (ULCS) is a powerful tool for cytogenetic analysis. As a proof of concept, we performed the analysis on 5 samples and a modified ULCS strategy was used for this study. Previous study indicated coefficient of variation (CV) in ULCS (<0.01-fold coverage) was lower than 15% on each autosome and there was no significant difference of the autosomal CVs between MiSeq and Ion Proton platforms. In a ULCS analysis, we assumed the UA on each chromosome (labeled as subscript i, i=1, 2, . . . , 22, X, Y) fits Poisson distribution.

UAᵢ=nᵢφᵢ

Where nᵢ is the number of reads needed to cover a chromosome i, and φᵢ is the coverage of a chromosome i. The percentage of UA on each chromosome (% UAᵢ) is determined by the length and copy number of each chromosome under the same coverage.

The lower limit of sequencing read needed for ULCS was primarily determined by the UA assigned to Chromosome Y because a) it is one of the shortest chromosomes, and thus fewer DNA fragments would be sequenced from it, b) less than 50% of chromosome Y has been sequenced and annotated in the human reference genome, and hence more than half of the Chromosome Y reads would not be able to be mapped to reference genome, and then being counted and c) reads mapped to the identical regions of the chromosome X and Y would not be considered as UA by the analysis pipeline. Moreover, crosslinking between chromosome X and Y, and the present of repetitive elements will cause a small portion of misplacement of reads from X and Y chromosome, which will further reduced reads that could have been mapped to the Y chromosome.

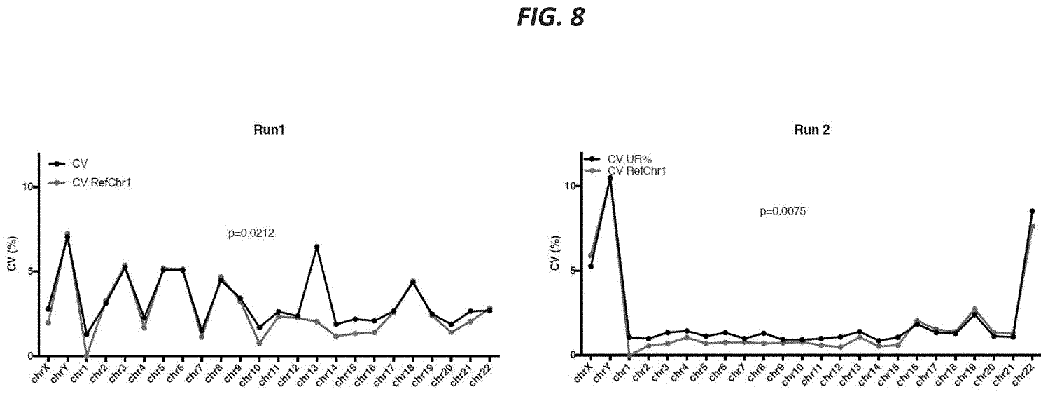

To estimate the lower limit of UAᵢ needed for ULCS cytogenetic analysis, we used Normal Approximation of Poisson distribution in R (qpois function) to estimate the detection power of UA for aneuploidy. It was estimated that the when UAᵢ=41, p(x>1.25λ)=0.04, p(x>1.5λ)=0.0008, and the detection power of aneuploidy is 90%. When the UAᵢ was 79, the detection power of aneuploidy would be 95.6%. The corresponding total UA for UA_{Y}˜79 is ˜15,000 in the normal male sample. 15,000 UA were randomly selected from the sequencing result of the normal male for 30 times, and the average UA for each chromosome was used as reference for normalization purpose (Ref_UAᵢ). To examine if the 15K reference is representing human genome under Poisson distribution, we compared the percentage of ungapped length (% UL) and % UA of each chromosome. Their ratios (Norm_Ref % UA) on autosomes was 1.04 (SD=0.0687, CV=6.6%) (FIG. 6).

The 15K reference represent the % UA represented about a half of the % UL of the sex chromosomes, which could be the result of depletion of non-unique alignments on homogenous regions of sex chromosomes. The mitochondrial chromosome (MT) is a multi-copy small chromosome, and it was not included in ULCS cytogenetics analysis. According to Poisson distribution, the 99.9% confidential intervals of each chromosome of the normal male reference can be estimated as Ref_UAᵢ±3.29\sqrt{Ref_UAᵢ} under the same coverage.

To access the copy number of each chromosome of a query sample using 15,000 UA reads (FIG. 7), we assumed the number of uniquely aligned read on each chromosome (UAᵢ) fits Poisson distribution as described before.

Using 15,000 UA reads, the normalized ratio between a query sample and the reference (Norm_% UAᵢ) was determined by the copy number of chromosomes:

$$Norm\_ \% {UA}_{i} = \frac{Query\_ \% {UA}_{i}}{Ref\_ \% {UA}_{i}} = \frac{{Query\_n}_{i} \times {Query\_φ}_{i}}{{Ref\_n}_{i} \times {Ref\_φ}_{i}}$$

To address the change in coverage y due to loss or gain of chromosomes, the corrected normalized % UAᵢ equals:

$${Norm}^{′} \_ \% {UA}_{i} = \frac{Norm\_ \% {UA}_{i}}{\overleftarrow{Norm\_ \% {UA}_{i}^{′}}}$$

Where [symbol] is the average Norm_% UAᵢ of normal autosomes as determined by Z-score. For an unknown sample, The standard deviation (SD) of Norm_% UAᵢ of normal autosomes (SDnormal) was estimated by known normal autosomes (within Ref_UAᵢ±3.29\sqrt{Ref_UAᵢ} ) in this study (n=105, SDnormal=0.0489). The Z-score was calculated for each chromosome:

$$Z - s c o r {e}_{i} = \frac{Norm\_ \% {UA}_{i} - Mean\_ \% {UA}_{autosome}}{S {D}_{normal}}$$

Chromosomes having a |Z-score| of >3.29 were considered as an abnormal chromosome with p<0.001. When the Z-score was >3.29, we consider there to be a gain of a chromosome, when the Z-score was <−3.29, we consider there to be a loss of a chromosome. While the modified Z-score method would be less specific in detecting abnormality on small autosomes than the Z-score method based on census of each chromosome, it provided sufficient detection power for aneuploidy detection (>95%) (FIG. 2C). The theoretical value of a normal autosome Norm′_% UAₙₒᵣₘₐₗ=1, a full trisomy of autosome Norm′_% UA_{trisomy}=1.5, a monosomy of autosome Norm′_% UA_{monosomy}=0.5, the X chromosome of a normal female Norm′_% UA_{x_female}>1.5, the Y chromosome of a normal female or missing Y chromosome Norm′_% UA_{y_female}<0.5.

We hypothesized that the corrected normalized % UAᵢ (Norm′_% UAᵢ) reflects the copy number of chromosomes. The Norm′_% UAᵢ were used to compute the adjusted Z-score (Z′-score). Norm′_% UAᵢ of normal autosomes with |Z-score|<3.29 were summarized (Mean_Norm′_% UA=0.9999, SD_Norm′_% UA=0.0481). Z′-score for each chromosome equals:

$${Z}^{′} - {score}_{i} = \frac{{Norm}^{′} \_ \% {UA}_{i} - {Mean\_Norm}^{′} \_ \% UA}{{SD\_Norm}^{′} \_ \% UA}$$

In brief, 15,000 UA were randomly selected from the normal male sample—and this was repeated for a total of 30 times—and averaged for normalization purpose (Ref_UA). For each sample, the first 15,000 UA (Query_UA) were selected for gender determination and aneuploidy detection. The UA were summarized and counted for each chromosome (UAᵢ=1, 2, . . . X, Y), and corresponding percentage were calculated for each chromosome (% UAᵢ) by UAᵢ/15,000×100. The % UAᵢ for each of the chromosome of a query sample (Query_% UAᵢ) was normalized to the normal male reference (Ref_% UAᵢ) and corrected to detect the copy number of each chromosome (Norm′_% UAᵢ) (FIG. 7 FIG. 2A).

### Example 8

### Internal Normalization

For determination of a copy number variation and/or aneuploidy using DNA sequencing or microarray, the signal abundance in a test samples is compared with the signal abundance in a reference sample. For example, when “X” ng of DNA from Test sample A is sequenced, 100 k unique reads map to Chromosome 21. When “X” ng of DNA from Test sample B is sequenced in the same sequencing run, 150 k unique reads map to Chromosome 21. However, when “X” ng of reference, normal, DNA sample is sequenced in the same sequencing run, 100 k unique reads are map to Chromosome 21. Thus Sample A has the same abundance of Chromosome 21 as does the reference sample while Sample B has 50% more, i.e. trisomy 21.

In another embodiment, the relative abundance of reads mapping to chromosome 21 are compared with an internal reference, such as chromosome 1. A normal ratio can be determined using a reference sample. In future runs, the ratio of reads from chromosome 1 relative to the number of reads from chromosome 21 would be determined. A decrease in this ratio would suggest a relative increase in the abundance of chromosome 21 relative to the reference chromosome.

This analysis can be done in conjunction with traditional analysis with a reference sample in order to improve the sensitivity and specificity of the test (e.g. low coverage sequencing or microarray) or it can be run alone in order avoid the need to also run a reference sample.

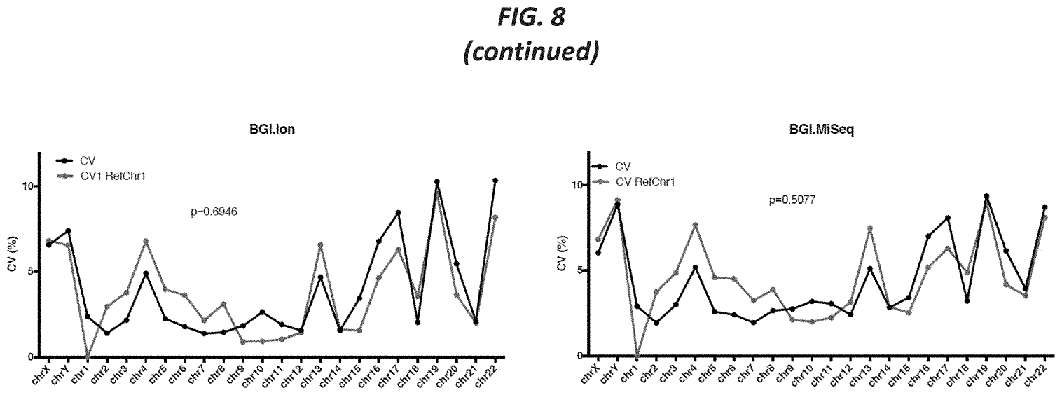

As shown in FIG. 8, Runs 1-4, using an internal reference has a very low coefficient of variation, whether using our own DNA sequencing data, or that obtained from other groups.

1-29. (canceled)

30. A method of preparing a nucleic acid sample for nanopore sequencing to determine chromosomal number, the method comprising:
(a) Obtaining a biological sample comprising genomic DNA from a subject;
(b) Isolating genomic DNA from the biological sample;
(c) Fragmenting the genomic DNA to a size of less than about-1000 base pairs per fragment using sonication;
(d) Modifying the fragmented genomic DNA to add dA tails to both ends of each DNA fragment;
(e) Purifying the modified fragmented DNA using bead purification;
(f) Concentrating the purified DNA into a reduced reaction volume;
(g) Further modifying the concentrated DNA to ligate a first adaptor to one end of the DNA and a second adaptor to the other end of the DNA, wherein the first adaptor comprises a Y-shaped adaptor, and the second adaptor comprises a hairpin-shaped adaptor;
(h) Performing nanopore sequencing of the prepared nucleic acid sample.

31. The method of claim 30, wherein determining chromosomal number detects aneuploidy or genetic sex.

32. The method of claim 30, wherein the Y-shaped adapter comprises an E5 protein and the hairpin-shaped adaptor comprises an E3 protein.

33. The method of claim 32, wherein the E3 protein comprises a His-Tag for purifying the DNA fragments ligated to the hairpin adaptor using His-Tag bead purification.

34. The method of claim 30, wherein the ligation step (g) comprises a prolonged 2-hour incubation at 4° C.

35. The method of claim 30, wherein the sequencing is done in real time.

36. The method of claim 30, wherein the sequencing is done in an office setting, or in a field setting, or in a clinical lab.

37. The method of claim 30, wherein the fragmented DNA is less than about 500 base pairs in length.

38. The method of claim 30, wherein the fragmented DNA is less than about 100 base pairs in length.

39. The method of claim 30, wherein preparation of the nucleic acid sample is performed in low-retention tubes.

40. The method of claim 30, wherein preparing a nucleic acid sample for nanopore sequencing to determine chromosomal number occurs within 2 to 4 hours.

41. The method of claim 30, wherein preparing a nucleic acid sample for nanopore sequencing to determine chromosomal number occurs within 1 to 2 hours.

42. The method of claim 30, further comprising comparing the sequence reads to a reference sample.

43. The method of claim 30, wherein abundance of the prepared nucleic acid sample determined by nanopore sequencing is compared with a reference sample.

44. The method of claim 43, wherein the reference sample comprises a human chromosome.

45. The method of claim 44, wherein the human chromosome comprises chromosome 1, chromosome 2, chromosome X, chromosome Y, or a portion thereof.

46. The method of claim 30, wherein the biological sample is selected from a tissue sample, products of conception, amniotic fluid, a chorionic villus biopsy, or maternal blood.

47. The method of claim 30, wherein the biologic sample comprises DNA extracted from one or more cells.

48. The method of claim 47, wherein the one or more cells comprises one or more blastomeres or blastocysts.

49. The method of claim 30, wherein PCR primers specific for each end of a target genomic sequence are used to amplify the target sequence, and wherein the nanopore sequencing identifies the presence or absence of the amplified target sequence.
